(19) United States
(12) Patent Application Publication
(10) Pub. No.: US 2002/0086276 A1
(43) Pub. Date: Jul. 4, 2002

(54) Immunotherapeutic methods for extracorporeal modulation of CD36 and its ligands

(76) Inventor: Pramod K. Srivastava, Avon, CT (US)

Correspondence Address: PENNIE AND EDMONDS, 1155 AVENUE OF THE AMERICAS, NEW YORK, NY, 100362711

(21) Appl. No.: 09/750,973

(22) Filed: Dec. 28, 2000

(51) Int. Cl. A61K 39/395

(52) U.S. Cl. 435/2; 424/140.1

(57) ABSTRACT

The present invention relates to methods for modulating an immune response in a patient by modulating the circulating levels of CD36 ligands. In particular, an extracorporeal apheresis method is described which can be used to modulate the levels of CD36 ligands in the bloodstream of a patient in need of immunotherapy.

### 1. INTRODUCTION

The present invention relates to methods for modulating an immune response in a patient by modulating the circulating levels of CD36 ligands. In particular, an extracorporeal apheresis method is described which can be used to modulate the levels of CD36 ligands in the bloodstream of a patient in need of immunotherapy.

### 2. BACKGROUND OF THE INVENTION

### 2.1. Heat Shock Proteins

Heat shock proteins (HSPs), also referred to as stress proteins, were first identified as proteins synthesized by cells in response to heat shock. Hsps have been classified into five families, based on molecular weight, Hsp100, Hsp90, Hsp70, Hsp60, and smHsp. Many members of these families were found subsequently to be induced in response to other stressful stimuli including nutrient deprivation, metabolic disruption, oxygen radicals, and infection with intracellular pathogens (see Welch, May 1993, Scientific American 5664; Young, 1990, Annu. Rev. Immunol. 8:401-420; Craig, 1993, Science 260:1902-1903; Gething et al., 1992, Nature 355:33-45; and Lindquist et al., 1988, Annu. Rev. Genetics 22:631-677).

Heat shock proteins are among the most highly conserved proteins in existence. For example, DnaK, the Hsp70 from E. coli has about 50% amino acid sequence identity with Hsp70 proteins from excoriates (Bardwell et al., 1984, Proc. Natl. Acad. Sci. 81:848-852). The Hsp60 and Hsp90 families also show similarly high levels of intra-family conservation (Hickey et al., 1989, Mol. Cell. Biol. 9:2615-2626; Jindal, 1989, Mol. Cell. Biol. 9:22792283). In addition, it has been discovered that the Hsp60, Hsp70 and Hsp90 families are composed of proteins that are related to the stress proteins in sequence, for example, having greater than 35% amino acid identity, but whose expression levels are not altered by stress.

Studies on the cellular response to heat shock and other physiological stresses revealed that the HSPs are involved not only in cellular protection against these adverse conditions, but also in essential biochemical and immunological processes in unstressed cells. HSPs accomplish different kinds of chaperoning functions. For example, members of the Hsp70 family, located in the cell cytoplasm, nucleus, mitochondria, or endoplasmic reticulum (Lindquist et al., 1988, Ann. Rev. Genetics 22:631-677), are involved in the presentation of antigens to the cells of the immune system, and are also involved in the transfer, folding and assembly of proteins in normal cells. HSPs are capable of binding proteins or peptides, and releasing the bound proteins or peptides in the presence of adenosine triphosphate (ATP) or low pH.

### 2.2. Immunogenicity of HSP-Peptide Complexes

Srivastava et al. demonstrated immune response to methylcholanthrene-induced sarcomas of inbred mice (1988, Immunol. Today 9:78-83). In these studies, it was found that the molecules responsible for the individually distinct immunogenicity of these tumors were glycoproteins of 96 kDa (gp96) and intracellular proteins of 84 to 86 kDa (Srivastava et al., 1986, Proc. Natl. Acad. Sci. USA 83:3407-3411; Ullrich et al., 1986, Proc. Natl. Acad. Sci. USA 83:3121-3125). Immunization of mice with gp96 or p84/86 isolated from a particular tumor rendered the mice immune to that particular tumor, but not to antigenically distinct tumors. Isolation and characterization of genes encoding gp96 and p84/86 revealed significant homology between them, and showed that gp96 and p84/86 were, respectively, the endoplasmic reticular and cytosolic counterparts of the same heat shock proteins (Srivastava et al., 1988, Immunogenetics 28:205-207; Srivastava et al., 1991, Curr. Top. Microbiol. Immunol. 167:109-123). Further, Hsp70 was shown to elicit immunity to the tumor from which it was isolated but not to antigenically distinct tumors. However, Hsp70 depleted of peptides was found to lose its immunogenic activity (Udono and Srivastava, 1993, J. Exp. Med. 178:1391-1396). These observations suggested that the heat shock proteins are not immunogenic per se, but form noncovalent complexes with antigenic peptides, and the complexes can elicit specific immunity to the antigenic peptides (Srivastava, 1993, Adv. Cancer Res. 62:153-177; Udono et al., 1994, J. Immunol., 152:5398-5403; Suto et al., 1995, Science, 269:1585-1588).

Noncovalent complexes of HSPs and peptide, purified from cancer cells, can be used for the treatment and prevention of cancer and have been described in PCT publications WO 96/10411, dated Apr. 11, 1996, and WO 97/10001, dated Mar. 20, 1997 (U.S. Pat. No. 5,750,119 issued Apr. 12, 1998, and U.S. Pat. No. 5,837,251 issued Nov. 17, 1998, respectively, each of which is incorporated by reference herein in its entirety). The isolation and purification of stress protein-peptide complexes has been described, for example, from pathogen-infected cells, and can be used for the treatment and prevention of infection caused by the pathogen, such as viruses, and other intracellular pathogens, including bacteria, protozoa, fungi and parasites (see, for example, PCT Publication WO 95/24923, dated Sep. 21, 1995, U.S. Pat. No. 6,048,530 issued Apr. 11, 2000). Immunogenic stress protein-peptide complexes can also be prepared by in vitro complexing of stress protein and antigenic peptides, and the uses of such complexes for the treatment and prevention of cancer and infectious diseases has been described in PCT publication WO 97/10000, dated Mar. 20, 1997 (U.S. Pat. No. 6,030,618 issued Feb. 29, 2000. The use of stress proteinpeptide complexes for sensitizing antigen presenting cells in vitro for use in adoptive immunotherapy is described in PCT publication WO 97/10002, dated Mar. 20, 1997 (see also U.S. Pat. No. 5,985,270 issued Nov. 16, 1999).

### 2.3. Antigen Presentation

Major histocompatibility complex (MHC) molecules present antigens on the cell surface of antigen-presenting cells. Cytotoxic T lymphocytes (CTLs) then recognize MHC molecules and their associated peptides and kill the target cell. Antigens are processed by two distinct antigen processing routes depending upon whether their origin is intracellular or extracellular. Intracellular or endogenous protein antigens, i.e., antigens synthesized within the antigen-presenting cell, are presented by MHC class I (MHC I) molecules to CD8+ cytotoxic T lymphocytes. On the other hand, extracellular or exogenously synthesized antigenic determinants are presented on the cell surface of “specialized” or “professional” APCs (macrophages, for example) by MHC class II molecules to CD4+ T cells (see, generally, Fundamental Immunology, W. E. Paul (ed.), New York: Raven Press, 1984). This compartmental segregation of antigen processing routes is important to prevent tissue destruction that could otherwise occur during an immune response as a result of shedding of neighboring cell MHC I antigens.

The heat shock protein gp96 chaperones a wide array of peptides, depending upon the source from which gp96 is isolated (for review, see Srivastava et al., 1998, Immunity 8:657665). Tumor-derived gp96 carries tumor-antigenic peptides (Ishii et al., 1999, J. Immunology 162:1303-1309); gp96 preparations from virus-infected cells carry viral epitopes (Suto and Srivastava, 1995, Science 269:1585-1588; Nieland et al., 1998, Proc. Natl. Acad. Sci. USA 95:1800-1805), and gp96 preparations from cells transfected with model antigens such as ovalbumin or β-galactosidase are associated with the corresponding epitopes (Arnold et al., 1995, J. Exp. Med.182:885-889; Breloer et al, 1998, Eur. J. Immunol. 28:1016-1021). The association of gp96 with peptides occurs in vivo (Menoret and Srivastava, 1999, Biochem. Biophys. Research Commun. 262:813-818). gp96-peptide complexes, whether isolated from cells (Tamura et al., 1997, Science 278:117-120), or reconstituted In vitro (Blachere et al., 1997, J. Exp. Med. 186:1183-1406) are excellent immunogens and have been used extensively to elicit CD8+ T cell responses specific for the gp96-chaperoned antigenic peptides.

The capacity of gp96-peptide complexes to elicit an immune response is dependent upon the transfer of the peptide to MHC class I molecules of antigen-presenting cells (Suto and Srivastava, 1995, supra). Endogenously synthesized antigens chaperoned by gp96 in the endoplasmic reticulum [ER] can prime antigen-specific CD8+ T cells (or MHC I-restricted CTLs) in vivo; this priming of CD8+ T cells requires macrophages. However, the process whereby exogenously introduced gp96-peptide complexes elicit the antigen-specific CD8+ T cell response is not completely understood since there is no established pathway for the translocation of extracellular antigens into the class I presentation machinery. Yet antigenic peptides of extracellular origin associated with HSPs are somehow salvaged by macrophages, channeled into the endogenous pathway, and presented by MHC I molecules to be recognized by CD8+ lymphocytes (Suto and Srivastava, 1995, supra; Blachere et al., 1997, J. Exp. Med. 186:1315-22).

Several models have been proposed to explain the delivery of extracellular peptides for antigen presentation. One proposal, known as the “direct transfer” model, suggests that HSP-chaperoned peptides are transferred to MHC I molecules on the cell surface of macrophages for presentation to CD8+ T lymphocytes. Another suggestion is that soluble extracellular proteins can be trafficked to the cytosol via constitutive macropinocytosis in bone marrow-derived macrophages and dendritic cells (Norbury et al., 1997, Eur. J. Immunol. 27:280-288). Yet another proposed mechanism is that HSPs are taken up by the MHC class I molecules of the macrophage, which stimulate the appropriate T cells (Srivastava et al., 1994, Immunogenetics 39:93-98). Others have suggested that a novel intracellular trafficking pathway may be involved for the transport of peptides from the extracellular medium into the lumen of ER (Day et al., 1997, Proc. Natl. Acad. Sci. 94:8064-8069; Nicchitta, 1998, Curr. Opin. in Immunol. 10:103-109). Further suggestions include the involvement of phagocytes which (a) possess an ill-defined pathway to shunt protein from the phagosome into the cytosol where it would enter the normal class I pathway; (b) digest ingested material in lysosomes and regurgitate peptides for loading on the surface to class I molecules (Bevan, 1995, J. Exp. Med. 182:639-41).

Still others have proposed a receptor-mediated pathway for the delivery of extracellular peptides to the cell surface of APCs for antigen presentation. In view of the extremely small quantity of gp96-chaperoned antigenic peptides required for immunization (Blachere et al., 1997, supra), and the strict dependence of immunogenicity of gp96-peptide complexes on functional antigen presenting cells (APCs) (Udono et al., 1994, Proc. Natl. Acad. Sci. U.S.A. 91:3077-3081), APCs had been proposed to possess receptors for gp96 (Srivastava et al., 1994, Immunogenetics 39:93-98). Such a receptor was recently identified and determined to be the alpha (2) macroglobulin receptor or CD91 (Binder et al., 2000, Nature Immunol. 1:151-155). It has been proposed that gp96 carries peptides into a cell via CD91 and transfers these peptides to the MHC class I molecules of dendritic and antigen presenting cells.

### 2.4. CD36 As a Heat Shock Protein Receptor

CD36 is a member of the class B scavenger receptor family and is primarily expressed in capillary endothelial cells, mammary secretory epithelial cells, differentiated adipose cells, B cells, macrophages, and several types of tumor cells. CD36 is believed to play a role in platelet adhesion and aggregation, phagocytosis of apoptotic cells, and in the metabolism of long-chain fatty acids. CD36 is located in the plasma membrane in microdomains called caveolae, which have been implicated in cellular transport and signaling pathways (Yamada et. al., 1998, Cell Mol. Life Sci. 54:628-640). Structurally, CD36 has a large (463 aa) extracellular domain, a single transmembrane domain and a short cytoplasmic tail containing a putative tyrosine kinase docking site. Amino acid residues #184-204 of CD36 make up a hydrophobic stretch of the receptor that is most likely associated with the plasma membrane. Anti-CD36 isoantibody recognizes a highly antigenic structure at amino acids 155-183 which is known as the immunodominant domain.

CD36 is expressed in both the monocytic and megakaryocytic lineage where it is upregulated during differentiation. Monocyte expression of CD36 is regulated by M-CSF and IL-4 and through adherence to activated endothelial cells (Yesner et al., 1996, Arterioscler. Thromb. Vasc. Biol. 16:1019-1025; Huh et al., 1995, J. Biol. Chem. 270:62676271). Expression of murine or human CD36 in CD36-deficient cells results in specific and high-affinity binding of oxidized LDL, followed by LDL internalization and degradation (Endemann et al., 1993, J. Biol. Chem. 268:11811-11816; Navazo et. al., 1996, Arterioscler. Thromb. Vasc. Biol. 16:1033-1039). CD36 also binds to long-chain fatty acids and its expression has been shown to be upregulated in endothelial cells in tissues involved in fattyacid transport and metabolism (Greenwalt et al., 1995, J. Clin. Invest. 96:1382-1388).

CD36 deficiency in humans is seen in approximately 3% of the Japanese population and 0.3% of Caucasians. Afflicted people are phenotypically normal but some subjects suffer from a reduced capacity to bind and internalize oxidized LDL (Nozaki et al., 1995, J. Clin. Invest. 96:1859-1865).

CD36 has also been shown to play a role in cytoadherence to Plasmodium falciparum-infected erythrocytes. After infection, P. falciparum become sequestered within the microvasculature, which helps contribute to the survival of the bacterium by preventing clearance in the spleen. CD36 binds to the PfEMP1 protein which is produced by P.falciparum in the infected erythrocyte. This binding can induce an oxidative burst of monocytes and platelet activation (Okenhouse et al., 1989, J. Clin. Invest. 84:468-475; Huang et. al., 1991, Proc. Natl. Acad. Sci. USA 88:7844-7848).

CD36 has also been shown to act as a receptor for heat shock proteins. In particular, the heat shock protein gp96 has been shown to bind to CD36 and stimulate the production of nitric oxide and chemokine in these cells. (Panjwani et. al., 2000, Cell Stress & Chaperones 5(4):373-397; U.S. Provisional Application No. 60/238865, filed on Oct. 6, 2000).

### 2.5. Extracorporeal Apheresis

The term “apheresis” is used to describe a procedure where blood is 1) withdrawn, 2) separated into fractions, and 3) at least one fraction retransfused back into the patient. Specific types of apheresis procedures include: “plasmapheresis” (for the collection of blood plasma), “leukapheresis” (for the collection of leukocytes), “thrombocytapheresis” (for the collection of platelets), therapeutic plasma exchange (wherein a portion of the subject's blood plasma is replaced with other fluids), and therapeutic plasma processing (a process where a portion of the patient's plasma is separated, treated or processed and then returned to the patient).

Originally, aphersis procedures were performed manually. This required blood to be withdrawn from the patient, treated appropriately in the lab, and then reinfused back into the patient. It was necessary to repeat such a procedure several times until the maximum allowable volume of plasma or other blood constituent had been collected. More recently, automated apheresis machines have been developed to minimize the amount of manual activity that is required in the handling and processing of the blood and its components. The automated apheresis machines normally include a central computer electrically connected to, and programmed to control, a system of tubes, vessels, filters, a peristaltic pump or tubing pump for moving blood, and usually a blood separation device.

Automated apheresis procedures require good venuous access with a blood flow of 50-100 ml/minute (Urbaniak and Robinson, 1990, BMJ 300(6725): 662-665). Various blood separators have been developed, some of which operate on a single vein systems. These separators are slower than the true continuous flow systems that require one vein for withdrawal and a second vein for return. All modern automated apheresis systems have safety features such as a pressure sensor alarm, microfilters, air detectors, and metered pumps.

Apheresis procedures can be carried out for either commercial purposes such as isolating commercially usable blood components or for therapeutic purposes. Therapeutic apheresis includes various procedures wherein a specific fraction of the blood is isolated and subjected to extracorporeal treatment, such as radiotherapy, chemotherapy, chelation therapy, or adsorptive removal of specific substances by passing the isolated blood fraction through an adsorptive column or the like. This form of therapy provides a means for removing abnormal blood constituents or by adding a blood constituents that help promote or stimulate a desired cellular action. Some conditions that have been managed by the use of therapeutic apheresis include: myasthenia gravis (Komfeld et.al., 1980, Plasma Ther., 2:127-133), multiple sclerosis (Weiner et. al., 1980, Neurology, 30:1029-1033), leukemia (Fay et. al., 1979, 54:747-749; Lane et. al., 1974, Medicine 53:463), and hemophilia (Spero et. al., 1980, Plasma Ther. 1:19-22). The use of therapeutic aphersis in treating these diseases includes removing unwanted cells or proteins from the blood such as an autoantibody in an autoimmune diseases or abnormal cellular fractions in sickle cell diseases and leukemias. Alternatively, therapeutic apheresis may be used to add a substance such as a cytokine or lymphokine to a population of lymphocytes in order to improve the effectiveness of treating a patient of diseases such as cancer or AIDS.

Citation or discussion of a reference herein shall not be construed as an admission that such is prior art to the present invention.

### 3. SUMMARY OF THE INVENTION

The present invention relates to methods for regulating CD36 ligands using extracorporeal methods for use in immunotherapy. The invention is based on the discovery by the Applicant that modulation of the levels of CD36 ligands in the bloodstream of a patient can result in stimulation of the immune response. Extracorporeal methods, described herein, are used to increase or decrease CD36 ligand levels in the blood for treatment of patients with immunodisorders, cancer and infectious diseases.

In another embodiment, a method is provided for stimulating an immune response in a patient comprising: a) removing a CD36 ligand from blood withdrawn from said patient; and b) returning at least a portion of the CD36 ligand-depleted blood to said patient.

In another embodiment, a method is provided for stimulating an immune response in a patient comprising: a) withdrawing blood from said patient; b) removing a CD36 ligand from said blood; and c) returning at least a portion of the CD36 ligand-depleted blood to said patient. In a specific embodiment, the method further comprises after step (a) and before step (c) the step of adding a heat shock protein or a heat shock protein antigenic-peptide complex to said blood. In a specific embodiment, said blood is returned to said patient by syringe. In another specific embodiment, said blood is returned to said patient by an intravenous drip. In another specific embodiment, the removing a CD36 ligand from the blood comprises the step of contacting the blood with a solid phase attached to a CD36 ligand-binding molecule for a time period and under conditions sufficient to allow binding of CD36 ligand to the CD36 ligand-binding molecule solid phase. In another specific embodiment, the CD36 ligand-binding molecule is CD36, or a fragment thereof. In another embodiment, said CD36 ligand-binding molecule does not bind a heat shock protein. In another embodiment, the CD36 ligand-binding molecule is an CD36 ligand-specific antibody, or a fragment thereof.

In various embodiments, an apheresis system is used in said removing step. In other embodiments blood is withdrawn manually in said withdrawing step. In various embodiments, said removing step comprises separating the plasma from said blood and treating said plasma to remove said CD36 ligand.

The invention further provides a kit comprising in one or more containers a solid phase chromatography column with a purified CD36 ligand binding molecule attached thereto, such that withdrawn blood can be run over the column to deplete the blood of a CD36 ligand. In one embodiment, the CD36 ligand binding molecule of the kit does not bind heat shock proteins.

The term “extracorporeal methods” or “extracorporeal procedures” as used herein refers to a process whereby blood is removed from a patient, treated appropriately, and then returned to the patient. Such procedures can be done either manually or by an apheresis machine.

The terms “HSP-CD36 related disorder” and “HSP-CD36 related condition”, as used herein, refer to disorders and conditions, respectively, involving a HSP-CD36 interaction. Such disorders and conditions may result, for example, from an aberrant ability of CD36 to interact with HSP, perhaps due to aberrant levels of HSP and/or CD36 expression, synthesis and/or activity relative to levels found in normal, unaffected, unimpaired individuals, levels found in clinically normal individuals, and/or levels found in a population whose levels represent a baseline, average HSP and/or CD36 levels. Such disorders include, but are not limited to, autoimmune disorders, diseases and disorders involving cellular signaling or growth disruption, diseases and disorders involving cytokine clearance and/or inflammation, proliferative disorders, viral disorders and other infectious diseases, hypercholesterolemia, Alzheimer's disease, diabetes, and osteoporosis.

The term “CD36 ligand” as used herein, refers to a molecule capable of binding to CD36. Such CD36 ligands include as well as known ligands, such as, but not limited to, lipoprotein complexes, thrombospondin 1, P. falciparum erythrocyte membrane protein 1 (PfEMP1), and phospholipids. In addition, CD36 ligands also include molecules which can readily be identified as CD36 ligands using standard binding assays well known in the art.

### 4. DETAILED DESCRIPTION OF THE INVENTION

The present invention relates to methods for modulating an immune response in a patient by altering the levels of CD36 ligands in the bloodstream using extracorporeal methods. The invention is based on the discovery by the Applicant that modulation of the levels of CD36 ligands in the bloodstream of a patient can result in stimulation of an immune response. CD36 acts as a heat shock protein receptor in CD36-expressing cells such as macrophages and dendritic cells. Binding of HSPs or HSP antigenic peptide complexes to such CD36-expressing cells results in stimulation of an immune response through the release of cytokines, chemokines, and nitric oxide. However, because CD36 is a scavenger receptor with diverse roles in different cell types and binds numerous non-HSP ligands, competition between CD36 ligands reduces the ability of HSPs and HSP complexes to access CD36.

The Applicant has discovered that depleting the blood of non-HSP-CD36 ligands and transfusing such CD36-ligand-depleted blood into the bloodstream of a patient can be used to stimulate the immune response, perhaps by increasing access of HSP complexes to CD36. Alternatively, blood can be depleted of CD36 ligands, including HSPs, followed by the addition of HSPs or HSP antigenic peptide complexes to stimulate a specific immune response. Decreasing the levels of competing CD36 ligands can be used to enhance a desired immune response in patients, such as patients with cancer and infectious disease. Such methods for depletion of CD36 ligands to the bloodstream are described in detail below.

The present invention uses compounds that interact with CD36 ligands such as antibodies, or CD36 itself, in extracorporeal procedures using affinity chromatography techniques to deplete the blood of these ligands. Depleting these ligands allows for heat shock proteins to bind to CD36 and stimulate an immune response.

### 4.1 Therapeutic Apheresis

Extracorporeal procedures described herein can be used to deplete non-HSP-CD36 ligands in the bloodstream of a patient in need of such treatment. Extracorporeal procedures, such as transfusion and apheresis, may be used to stimulate an immune response by modulating non-HSP-CD36 ligand levels in a patient's circulation or, alternatively, depleting CD36 ligands including HSPs from the blood, followed by the selective addition of specific HSPs or HSP antigenic peptide complexes to the blood. For example, in one embodiment, apheresis techniques coupled with affinity column technology are used to remove CD36 ligand from a patient's blood, followed by the return of the ligand-depleted blood into circulation.

In another embodiment, apheresis techniques coupled with affinity chromatography techniques are used to remove CD36 ligand from a patient's blood followed by the selective addition of HSPs or HSP antigenic peptide complexes to the patient's blood, and return of the treated blood into the patient's circulation.

Extraction of blood can be performed either manually or by any one of the common automated, electronically controlled “apheresis” systems, such as the Autopheresis-C.RTM. system (Baxter Healthcare Corporation, Fenwal Division, 1425 Lake Cook Road, Deerfield, Ill. 60015). In a preferred embodiment, a blood separation apparatus is fluidly connected to a blood vessel of the patient by way of a blood extraction tube. A blood pump, such as a peristaltic pump, is positioned on the blood extraction tube to pump blood from the patient to a blood separation apparatus. An anticoagulant, such as heparin, can be added to the blood through a separate chamber that is in fluid communication with the apheresis system.

Optionally, blood can be taken out of the apheresis system, treated to remove a CD36 ligand in the laboratory, and then put back into the apheresis system to be reintroduced to the patient. In another embodiment, the blood can be further separated into cellular components such that only a specific subset of cells (i.e. leukocytes) can be treated to remove a CD36 ligand and returned to the patient or, alternatively, only the plasma can be treated to remove a CD36 ligand and returned to the patient. In another embodiment, after the blood has been treated to remove a CD36 ligand, HSPs are added back to the blood.

In various embodiments, blood from a patient can be withdrawn manually and the cells can be separated by a standard laboratory blood cell collection device. After or during the cellular collection, the blood can be treated to remove a CD36 ligand. The cells can then be returned to the patient by an i.v. drip or by injection with a syringe.

In one embodiment, apheresis methods may be used to enhance an immune response. CD36 ligands are removed from transfused blood of a patient in need of treatment for an immune disorder. In another embodiment, the CD36 ligand that is removed from the blood is not a heat shock protein.

One example of such a method comprises the following steps: (1) withdrawing blood from a patient; (2) passing the patient's blood over an affinity column comprising a CD36 ligand-binding compound, such as an antibody specific for a non-HSP-CD36 ligand, for a time period and under conditions sufficient to allow binding of CD36 ligand to the affinity column; (3) returning the CD36-ligand depleted blood to the patient.

In another embodiment, apheresis methods may be used to enhance an immune response by depleting CD36 ligands (including HSPs) followed by the addition of selective HSPs or HSP antigenic peptide complexes to the blood of a patient.

An example of such a method comprises the following steps: (1) withdrawing blood from a patient; (2) passing the patient's blood over an affinity column comprising a CD36 ligand-binding compound for a time period and under conditions sufficient to allow binding of CD36 ligand to the affinity column; (3) adding HSPs or HSP antigenic peptide complexes to the ligand depleted blood; (4) returning the blood to the patient.

Methods that can be used to remove a ligand from the blood include affinity chromatography, anion or cation exchange chromatography, phosphocellulose chromatography, immunoaffinity chromatography, hydroxyapatite chromatography, and lectin chromatography. Affinity purification is based on the interaction between the compound on the affinity column and its binding partner. The principle of affinity chromatography is well known in the art. In one embodiment, a recombinantly expressed and purified (or partially purified) protein, such as CD36, is covalently or non-covalently coupled to a solid support such as, for example, a chromatography column. The extracted blood from a patient can be run over such a column. The coupled protein will bind to the CD36 ligand and deplete the blood of the CD36 ligand. The depleted blood can then be returned to the patient. In another embodiment, an antibody specific to the ligand can be coupled to the chromatography column and the immunospecific binding of an antibody to the CD36 ligand can be used to deplete the blood of the CD36 ligand. Alternatively, one of the many cation or anion exchange resins commonly used in the art can be used to deplete the blood of the non-HSP-CD36 ligand.

In another embodiment, the present invention also includes a kit that comprises a solid phase chromatography column with a purified CD36 ligand binding molecule attached thereto. Such a kit can contain components necessary for extracorporeal removal of CD36 ligands from the blood of a patient in need of such,treatment.

Transfusion/apheresis methods may also be used in combination with other methods of immunotherapy. In one embodiment, for example, after depletion of non-HSP CD36 ligands as described above, HSP-antigenic peptide complexes may be delivered to a cancer patient, or a patient having an infectious disease, using the transfusion/apheresis methods, or other method. Using transfusion/apheresis, at the same time as HSP-antigenic peptide complexes are being delivered, CD36 ligands (other than HSPs) may be removed from the patient's blood, in order to stimulate the immune response against the HSP-antigenic peptide complex being delivered. Thus, the transfusion/apheresis method makes it possible to accomplish both the delivery of HSP-antigenic peptide complexes and the removal of competing CD36 ligands in a single procedure.

### 4.2 Compounds for Use in Thereapeutic Apheresis

Compositions used in extracorporeal apheresis include CD36-ligand binding molecules. The term “CD36 ligand binding molecule” as used herein, refers to a molecule capable of binding to a CD36 ligand. Such CD36 ligand binding molecules include molecules that bind to CD36 ligands, such as, but not limited to, lipoprotein complexes, thrombospondin 1, P. falciparum erythrocyte membrane protein 1 (PfEMP1), LDL, and phospholipids. In addition, CD36 ligand binding molecules include molecules which can readily be identified to bind to CD36 ligands using standard binding assays well known in the art. Compounds or molecules that interact with CD36 or other CD36 ligand binding molecules, such as those identified by screening methods provided herein, are used in extracorporeal methods to treat HSP-CD36 related disorders and conditions, such as autoimmune diseases, cancer and infectious diseases.

CD36 ligand binding molecules are used in the present invention. Such molecules can be attached to a chromatography column and used in extracorporeal methods to deplete the blood of CD36 ligands. The molecules that bind CD36 ligands for use in the invention can be purified from natural sources, chemically synthesized, or recombinantly produced. Such molecules can be synthesized and be used to bind CD36 ligands using extracorporeal methods. In a preferred embodiment, the CD36 ligand binding molecule is mammalian (e.g., mouse, rat, primate, domestic animal such as dog, cat, cow, horse), and is most preferably, human. In addition, CD36 ligand binding molecule analogs, muteins, derivatives, and fragments can be used in place of CD36 ligand binding molecules

In one embodiment, the molecule that binds to a CD36 ligand is used in extracorporeal methods by attaching such molecule to a solid phase surface for use as an affinity chromatography column, such that blood can be run over the column to deplete the blood of the CD36 ligand. Such a depletion of a CD36 ligand will allow greater access of CD36 to heat shock proteins to stimulate an immune response.

In another embodiment, an antibody specific for the CD36 ligand or a fragment thereof which contains the binding site for the CD36 ligand is used in extracorporeal methods,

In another embodiment, the CD36 ligand binding molecule is a peptide which comprises at least 10 contiguous amino acids of CD36 sequence, which can bind to the CD36 ligand. In yet another embodiment, the CD36 ligand binding molecule is a peptide which comprises at least 10 contiguous amino acids of CD36 sequence, in particular the ECD of CD36 (or a portion thereof), which can bind to the CD36 ligands.

Such peptides may be produced synthetically or by using standard molecular biology techniques. Amino acid sequences and nucleotide sequences of naturally occurring CD36 ligands are generally available in sequence databases, such as GenBank. Computer programs, such as Entrez, can be used to browse the database, and retrieve any amino acid sequence and genetic sequence data of interest by accession number. Databases can also be searched to identify sequences with various degrees of similarities to a query sequence such as the sequence of known CD36 ligands using programs, such as FASTA and BLAST, which rank the similar sequences by alignment scores and statistics.

In a specific mode of the embodiment, the CD36 ligand binding molecule consists of at least 10, more preferably at least 20, yet more preferably at least 30, yet more preferably at least 40, and most preferably at least 80 (continuous) amino acids of CD36. In specific modes of the embodiment, such fragments are not larger than 40-45 amino acids. In other specific modes of the embodiment, such fragments are not larger than 80-90 amino acids.

Derivatives or analogs of CD36 can be used to bind to CD36 ligands for use in extracorporeal methods. Such derivative or analogs include but are not limited to those molecules comprising regions that are substantially homologous to the extracellular domain of CD36 or fragments thereof (e.g., in various embodiments, at least 60% or 70% or 80% or 90% or 95% identity over an amino acid sequence of identical size or when compared to an aligned sequence in which the alignment is done by a computer homology program known in the art) or whose encoding nucleic acid is capable of hybridizing to a sequence encoding a CD36 ligand-binding sequence, under stringent, moderately stringent, or nonstringent conditions. In certain specific embodiments, a CD36 derivative is a chimeric or fusion protein comprising a non-HSP-CD36 ligand binding domain of CD36, preferably consisting of at least one complement repeat of C1-II joined at its amino- or carboxy-terminus via a peptide bond to an amino acid sequence of a different protein. Such a chimeric protein can be produced recombinantly as described above, by omitting the cleavage repurification steps.

Other CD36 derivatives can be made by altering CD36 coding sequences by substitutions, additions or deletions that provide for functionally equivalent molecules. Due to the degeneracy of nucleotide coding sequences, other DNA sequences which encode substantially the same amino acid sequence as CD36 gene or gene fragment may be used in the practice of the present invention.

Peptides may be produced synthetically or by using standard molecular biology techniques. Amino acid sequences and nucleotide sequences of naturally occurring CD36 and CD36 ligand binding molecules are generally available in sequence databases, such as GenBank. Computer programs, such as Entrez, can be used to browse the database, and retrieve any amino acid sequence and genetic sequence data of interest by accession number. Methods for recombinant and synthetic production of such peptides are described in Sections 4.4 and 4.5.

Some other compositions needed to perform the invention include antibodies that specifically recognize non-HSP-CD36 ligands to be attached to a chromatography column to deplete ligands from withdrawn blood. In one embodiment, CD36 ligands other than HSPs are used for the production of antibodies against these ligands for use in extracorporeal procedures. The antibodies produced can be attached to a chromatography column and deplete the non-HSP-CD36 ligand from the blood. In a preferred embodiment, the CD36 ligand is mammalian (e.g., mouse, rat, primate, domestic animal such as dog, cat, cow, horse), and is most preferably, human. In addition, CD36 ligand analogs, muteins, derivatives, and fragments can be used in place of CD36 ligands.

In addition, isolated and recombinant cells that contain recombinant CD36 and CD36 ligand binding molecule sequences may be needed to perform the invention.

### 4.3 HSPS and HSP Antigentic Peptide Complexes

HSPs and HSP antigenic peptide complexes may be used in conjunction with the extracorporeal methods to add back HSP and HSP complexes to the CD36 ligand depleted blood.

An HSP useful in the practice of the invention may be selected from among any cellular protein that satisfies any one of the following criteria: the intracellular concentration of an HSP increases when a cell is exposed to a stressful stimulus; an HSP can bind other proteins or peptides, and can release the bound proteins or peptides in the presence of adenosine triphosphate (ATP) or low pH; or an HSP possesses at least 35% homology with any cellular protein having any of the above properties. Preferably, the HSP used in the compositions and methods of the present invention includes, but are not limited to, Hsp90, gp96, BiP, Hsp70, DnaK, Hsc70, PhoE calreticulin, PDI, or an sHsp, alone or in combination.

In a preferred embodiment, an HSP is mammalian (e.g., mouse, rat, primate, domestic animal such as dog, cat, cow, horse), and is most preferably, human.

Hsps useful in the practice of the invention include, but are not limited to, members of the Hsp60 family, Hsp70 family, Hsp90 family, HSP100 family, sHSP family, calreticulin, PDI, and other proteins in the endoplasmic reticulum that contain thioredoxinlike domain(s), such as, but not limited to, ERp72 and ERp61.

HSP analogs, muteins, derivatives, and fragments can also be used in place of HSPs according to the invention. An HSP peptide-binding “fragment” for use in the invention refers to a polypeptide comprising a HSP peptide-binding domain that is capable of becoming non-covalently associated with a peptide to form a complex that is capable of eliciting an immune response. In one embodiment, an HSP peptide-binding fragment is a polypeptide comprising an HSP peptide-binding domain of approximately 100 to 200 amino acids.

Databases can also be searched to identify sequences with various degrees of similarities to a query sequence using programs, such as FASTA and BLAST, which rank the similar sequences by alignment scores and statistics. Such nucleotide sequences of non-limiting examples of HSPs that can be used for preparation of the HSPs used in the methods of the invention are as follows: human Hsp70, Genbank Accession No. NM_{—}005345, Sargent et al., 1989, Proc. Natl. Acad. Sci. U.S.A., 86:1968-1972; human Hsp90, Genbank Accession No. X15183, Yamazaki et al., Nucl. Acids Res. 17:7108; human gp96: Genbank Accession No. X15187, Maki et al., 1990, Proc. Natl. Acad Sci., 87: 5658-5562; human BiP: Genbank Accession No. M19645; Ting et al., 1988, DNA 7: 275-286; human Hsp 27, Genbank Accession No. M24743; Hickey et al., 1986, Nucleic Acids Res. 14:4127-45; mouse Hsp70: Genbank Accession No. M35021, Hunt et al, 1990, Gene, 87:199-204; mouse gp96: Genbank Accession No. M16370, Srivastava et al., 1987, Proc. Natl. Acad. Sci., 85:3807-3811; and mouse BiP: Genbank Accession No. U16277, Haas et al., 1988, Proc. Natl. Acad. Sci. U.S.A., 85: 2250-2254. Due to the degeneracy of the genetic code, the term “HSP sequence”, as used herein, refers not only to the naturally occurring amino acid and nucleotide sequence but also encompasses all the other degenerate sequences that encode the HSP.

The aforementioned HSP families also contain proteins that are related to HSPs in sequence, for example, having greater than 35% amino acid identity, but whose expression levels are not altered by stress. Therefore, it is contemplated that the definition of heat shock or stress protein, as used herein, embraces other proteins, mutants, analogs, and variants thereof having at least 35% to 55%, preferably 55% to 75%, and most preferably 75% to 85% amino acid identity with members of these families whose expression levels in a cell are enhanced in response to a stressful stimulus. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul, et al., 1990, J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain 30 amino acid sequences homologous to a protein molecules of the invention. To obtain gapped alignments for comparison purposes, Gapped BLAST can be utilized as described in Altschul et al., 1997, Nucleic Acids Res.25:3389-3402. Alternatively, PSI-Blast can be used to perform an iterated search which detects distant relationships between molecules (Altschul et al., 1997, supra). When utilizing BLAST, Gapped BLAST, and PSI-Blast programs, the default parameters of the respective programs (e.g., XBLAST and NBLAST) can be used (see http://www.ncbi.nlm.nih.gov). Another preferred, non-limiting example of a mathematical algorithm utilized for the comparison of sequences is the algorithm of Myers and Miller, 1988, CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The HSPs, for use in the invention can be purified from natural sources, chemically synthesized, or recombinantly produced. Although the HSPs may be allogeneic to the patient, in a preferred embodiment, the HSPs are autologous to the patient to whom they are administered.

Methods for isolating and purifying HSPs have been described previously (see, for example, PCT publication WO 97/10000, dated Mar. 20, 1997, which is U.S. Pat. No. 6,030,618 issued Feb. 29, 2000; PCT publications WO 96/10411, dated Apr. 11, 1996, and WO 97/10001, dated Mar. 20, 1997 which is U.S. Pat. No. 5,750,119 issued Apr. 12, 1998, and U.S. Pat. No. 5,837,251 issued Nov. 17, 1998, respectively, each of which is incorporated by reference herein in its entirety). The isolation and purification of stress protein-peptide complexes has been described, for example, from pathogen-infected cells, and can be used for the treatment and prevention of infection caused by the pathogen, such as viruses, and other intracellular pathogens, including bacteria, protozoa, fungi and parasites (see, for example, PCT Publication WO 95/24923, dated Sep. 21, 1995, U.S. Pat. No. 6,048,530 issued Apr. 11, 2000).

The methods of the invention can be used, for example, in extracorporeal immunotherapy against proliferative disorders, infectious diseases, and other HSP-CD36-related disorders. Methods for the synthesis and production of such compositions are described herein.

### 4.4 Recombinant Expression

In various embodiments of the invention, sequences encoding CD36, or CD36 ligand binding molecules are inserted into an expression vector for propagation and expression in recombinant cells. Thus, in one embodiment, CD36 or a CD36 ligand binding molecule ligand coding region is linked to a non-native promoter for expression in recombinant cells.

In various embodiments of the invention, sequences encoding CD36, and/or a CD36 ligand binding molecule or fragments thereof, are inserted into an expression vector for propagation and expression in recombinant cells. An expression construct, as used herein, refers to a nucleotide sequence encoding a particular gene product, such as CD36 or a CD36 ligand binding molecule, operably associated with one or more regulatory regions which allows expression of the encoded gene product in an appropriate host cell. “Operably-associated” refers to an association in which the regulatory regions and the nucleotide sequence encoding the gene product to be expressed are joined and positioned in such a way as to permit transcription, and ultimately, translation.

The DNA may be obtained from known sequences derived from sequence databases by standard procedures known in the art by DNA amplification or molecular cloning directly from a tissue, cell culture, or cloned DNA (e.g., a DNA “library”). Any eukaryotic cell may serve as the nucleic acid source for obtaining the coding region of CD36 or a CD36 ligand binding molecule. Nucleic acid sequences encoding CD36 ligand binding molecules can be isolated from vertebrate, mammalian, as well as primate sources, including humans. Clones derived from genomic DNA may contain regulatory and intron DNA regions in addition to coding regions; clones derived from cDNA will contain only exon sequences. Whatever the source the CD36 or CD36 ligand binding molecule gene should be cloned into a suitable vector for propagation of the gene.

Vectors based on E. coli are the most popular and versatile systems for high level expression of foreign proteins (Makrides, 1996, Microbiol Rev, 60:512-538). Non-limiting examples of regulatory regions that can be used for expression in E. coli may include but not limited to lac, trp, Ipp, phoA, recA, tac, ηPL, and phage T3 and T7 promoters (Makrides, 1996, Microbiol Rev, 60:512-538). Non-limiting examples of prokaryotic expression vectors may include the ηgt vector series such as ηgt11 (Huynh et al., 1984 in “DNA Cloning Techniques”, Vol. 1: A Practical Approach (D. Glover, ed.), pp.49-78, IRL Press, Oxford), and the pET vector series (Studier et al., 1990, Methods Enzymol., 185:60-89). However, a potential drawback of a prokaryotic host-vector system is the inability to perform many of the post-translational processing events of mammalian cells. Thus, an eukaryotic host-vector system is preferred, a mammalian host-vector system is more preferred, and a human hostvector system is the most preferred.

The regulatory regions necessary for transcription of a CD36 sequence or a CD36 ligand binding molecule can be provided by the expression vector. A translation initiation codon (ATG) may also be provided to express a nucleotide sequence encoding CD36 or a CD36 ligand binding molecule that lacks an initiation codon. In a compatible host-construct system, cellular proteins required for transcription, such as RNA polymerase and transcription factors, will bind to the regulatory regions on the expression construct to effect transcription of the CD36 or CD36 ligand binding molecule sequence in the host organism. The precise nature of the regulatory regions needed for gene expression may vary from host cell to host cell. Generally, a promoter is required which is capable of binding RNA polymerase to initiate the transcription of an operably-associated nucleic acid sequence. Such regulatory regions may include those 5′-non-coding sequences involved with initiation of transcription and translation, such as the TATA box, the cap site, a CAAT box, and the like. The non-coding region 3′ to the coding sequence may contain transcriptional termination regulatory sequences, such as terminators and polyadenylation sites.

Both constitutive and inducible regulatory regions may be used for expression of CD36 or other CD36 ligand binding molecules. It may be desirable to use inducible promoters when the conditions optimal for growth of the recombinant cells and the conditions for high level expression of the gene product are different. Examples of useful regulatory regions are provided in the next section below.

For expression of CD36 or other CD36 ligand binding molecule gene products in mammalian host cells, a variety of regulatory regions can be used, for example, the SV40 early and late promoters, the cytomegalovirus (CMV) immediate early promoter, and the Rous sarcoma virus long terminal repeat (RSV-LTR) promoter. Inducible promoters that may be useful in mammalian cells include but are not limited to those associated with the metallothionein II gene, mouse mammary tumor virus glucocorticoid responsive long terminal repeats (MMTV-LTR), the β-interferon gene, and the Hsp70 gene (Williams et al., 1989, Cancer Res. 49:2735-42; Taylor et al., 1990, Mol. Cell Biol., 10:165-75). It may be advantageous to use heat shock promoters or stress promoters to drive expression of CD36 or a CD36 ligand binding molecule in recombinant host cells.

The following animal regulatory regions, which exhibit tissue specificity and have been utilized in transgenic animals, can also be used in tumor cells of a particular tissue type: elastase I gene control region which is active in pancreatic acinar cells (Swift et al., 1984, Cell 38:639-646; Omitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283286), and gonadotropic releasing hormone gene control region which is active in the hypothalamus (Mason et al., 1986, Science 234:1372-1378).

The efficiency of expression of CD36 or CD36 ligand binding molecule in a host cell may be enhanced by the inclusion of appropriate transcription enhancer elements in the expression vector, such as those found in SV40 virus, Hepatitis B virus, cytomegalovirus, immunoglobulin genes, metallothionein, β-actin (see Bittner et al., 1987, Methods in Enzymol. 153:516-544; Gorman, 1990, Curr. Op. in Biotechnol. 1:36-47).

The expression vector may also contain sequences that permit maintenance and replication of the vector in more than one type of host cell, or integration of the vector into the host chromosome. Such sequences may include but are not limited to replication origins, autonomously replicating sequences (ARS), centromere DNA, and telomere DNA. It may also be advantageous to use shuttle vectors that can be replicated and maintained in at least two types of host cells.

In addition, the expression vector may contain selectable or screenable marker genes for initially isolating or identifying host cells that contain DNA encoding CD36 or CD36 ligand binding molecules. For long term, high yield production of CD36 or CD36 ligand binding molecule, stable expression in mammalian cells is preferred. A number of selection systems may be used for mammalian cells, including, but not limited, to the herpes simplex virus thymidine kinase (Wigler et al., 1977, Cell 11:223), hypoxanthine-guanine phosphoribosyltransferase (Szybalski and Szybalski, 1962, Proc. Natl. Acad. Sci. USA 48:2026), and adenine phosphoribosyltransferase (Lowy et al., 1980, Cell 22:817) genes can be employed in tk⁻, hgprt⁻ or aprt⁻ cells, respectively. Also, antimetabolite resistance can be used as the basis of selection for dihydrofolate reductase (dhfr), which confers resistance to methotrexate (Wigler et al., 1980, Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neomycin phosphotransferase (neo), which confers resistance to the aminoglycoside G-418 (Colberre-Garapin et al., 1981, J. Mol. Biol. 150:1); and hygromycin phosphotransferase (hyg), which confers resistance to hygromycin (Santerre et al., 1984, Gene 30:147). Other selectable markers, such as but not limited to histidinol and Zeocinum™ can also be used.

In order to insert the DNA sequence encoding CD36 or CD36 ligand binding molecules into the cloning site of a vector, DNA sequences with regulatory functions, such as promoters, must be attached to DNA sequences encoding CD36 or other CD36 ligands, respectively. To do this, linkers or adapters providing the appropriate compatible restriction sites may be ligated to the ends of cDNA or synthetic DNA encoding CD36 or a CD36 ligand binding molecule, by techniques well known in the art (Wu et al., 1987, Methods in Enzymol 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA by use of PCR with primers containing the desired restriction enzyme site.

In one embodiment, an expression construct comprising CD36 sequence operably associated with regulatory regions can be directly introduced into appropriate host cells for expression and production of CD36 without further cloning (see, for example, U.S. Pat. No. 5,580,859). The expression constructs may also contain DNA sequences that facilitate integration of CD36 or a CD36 ligand binding molecule sequence into the genome of the host cell, e.g., via homologous recombination. In this instance, it is not necessary to employ an expression vector comprising a replication origin suitable for appropriate host cells in order to propagate and express CD36 in the host cells.

Expression constructs containing cloned nucleotide sequence encoding CD36 or a CD36 ligand binding molecule, can be introduced into the host cell by a variety of techniques known in the art, including but not limited to, for prokaryotic cells, bacterial transformation (Hanahan, 1985, in DNA Cloning, A Practical Approach, 1:109-136), and for eukaryotic cells, calcium phosphate mediated transfection (Wigler et al., 1977, Cell 11:223-232), liposome-mediated transfection (Schaefer-Ridder et al., 1982, Science 215:166-168), electroporation (Wolff et al., 1987, Proc Natl Acad Sci 84:3344), and microinjection (Cappechi, 1980, Cell 22:479-488).

For long term, high yield production of properly processed CD36 or CD36 ligand binding molecule stable expression in mammalian cells is preferred. Cell lines that stably express CD36 or a CD36 ligand binding molecule may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while the desired gene product is expressed continuously.

The recombinant cells may be cultured under standard conditions of temperature, incubation time, optical density, and media composition. Alternatively, recombinant antigenic cells may be cultured under conditions emulating the nutritional and physiological requirements of the cancer cell or infected cell. However, conditions for growth of recombinant cells may be different from those for expression of CD36 or CD36 ligand inding molecules.

### 4.5 Peptide Synthesis

Peptide synthesis is an alternative method to produce CD36 or CD36 ligand binding molecules. For example, a peptide corresponding to a portion of a CD36 ligand binding molecule or a CD36 peptide can be synthesized by use of a peptide synthesizer and attached to an affinity column to bind a CD36 ligand. Synthetic peptides corresponding to CD36 sequences useful for therapeutic methods described herein can also be produced synthetically. Conventional peptide synthesis may be used or other synthetic protocols well known in the art.

For example, peptides having the amino acid sequence of the CD36 or CD36 ligand binding molecules, or an analog, mutein, fragment, or derivative thereof, may be synthesized by solid-phase peptide synthesis using procedures similar to those described by Merrifield, 1963, J. Am. Chem. Soc., 85:2149. During synthesis, N-α-protected amino acids having protected side chains are added stepwise to a growing polypeptide chain linked by its C-terminal and to an insoluble polymeric support i.e., polystyrene beads. The peptides are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc which is acid labile and Fmoc which is base labile. Details of appropriate chemistries, resins, protecting groups, protected amino acids and reagents are well known in the art and so are not discussed in detail herein (See, Atherton, et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting CD36 or CD36 ligand binding molecule peptides is accomplished using conventional procedures, such as preparative HPLC using gel permeation, partition and/or ion exchange chromatography. The choice of appropriate matrices and buffers are well known in the art and so are not described in detail herein.

In addition, analogs and derivatives of CD36 or other CD36 ligand binding molecules can be chemically synthesized. Furthermore, if desired, nonclassical amino acids or chemical amino acid analogs can be introduced as a substitution or addition into CD36 or other CD36 ligand binding molecules. Non-classical amino acids include but are not limited to the D-isomers of the common amino acids, α-amino isobutyric acid, 4-aminobutyric acid, bu, 2-amino butyric acid, γ-Abu, ε-Ahx, 6-amino hexanoic acid, Aib, 2-amino isobutyric acid, 3-amino propionic acid, omithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general.

### 4.6 Antibodies Specific for CD36 Ligands

Described herein are methods for the production of antibodies capable of specifically recognizing CD36 ligands, such as non-HSP-CD36 ligands. Such antibodies can be used in the present invention by attaching the antibodies to a solid phase affinity column to deplete CD36 ligands from the blood. Extracted blood can be run through the column using extracorporeal procedures deplete the blood of the specific ligand. In one embodiment, an antibody against non-HSP-CD36 ligands is used to specifically deplete the blood of ligands which compete with HSP for binding to CD36.

Such antibodies may include, but are not limited to, polyclonal antibodies, monoclonal antibodies (mAbs), humanized or chimeric antibodies, single chain antibodies, Fab fragments, F(ab′)₂ fragments, fragments produced by a Fab expression library, antiidiotypic (anti-Id) antibodies, and epitope-binding fragments of any of the above.

For the production of antibodies against a CD36 ligand, various host animals may be immunized by injection with a CD36 ligand or a portion thereof. An antigenic portion of CD36 or a CD36 ligand can be readily predicted by algorithms known in the art.

Host animals may include, but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen or an antigenic functional derivative thereof. For the production of polyclonal antibodies, host animals such as those described above, may be immunized by injection with a CD36 ligand, or a portion thereof, supplemented with adjuvants as also described above.

Monoclonal antibodies, which are homogeneous populations of antibodies to a particular antigen, may be obtained by any technique that provides for the production of antibody molecules by continuous cell lines in culture. These include, but are not limited to, the hybridoma technique of Kohler and Milstein, (1975, Nature 256, 495-497; and U.S. Pat. No. 4,376,110), the human B-cell hybridoma technique (Kosbor et al., 1983, Immunology Today 4: 72; Cole et al., 1983, Proc. Natl. Acad. Sci. USA 80,2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison, et al., 1984, Proc. Natl. Acad. Sci., 81: 6851-6855; Neuberger, et al., 1984, Nature 312: 604-608; Takeda, et al., 1985, Nature, 314: 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region (see, e.g., Cabilly et al., U.S. Pat. No. 4,816,567; and Boss et al., U.S. Pat. No. 4,816,397, which are incorporated herein by reference in their entirety).

In an additional embodiment of the invention, monoclonal antibodies can be produced in germ-free animals (see PCT International Publication No. WO 89/12690, published Dec. 12, 1989). According to the invention, human antibodies may be used and can be obtained by using human hybridomas (Cote et al, 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96). Techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for CD36-HSP complex together with genes from a human antibody molecule of appropriate biological activity can also be used; such antibodies are within the scope of this invention.

Humanized antibodies are also provided (see U.S. Pat. No. 5,225,539 by Winter). An immunoglobuin light or heavy chain variable region consists of a “framework” region interrupted by three hypervariable regions, referred to as complementarity determining regions (CDRs). The extent of the framework region and CDRs have been precisely defined (see, “Sequences of Proteins of Immunological Interest”, Kabat, E. et al., U.S. Department of Health and Human Services (1983)). Briefly, humanized antibodies are antibody molecules from non-human species having one or more CDRs from the non-human species and a framework region from a human immunoglobulin molecule. Such CDRs-grafted antibodies have been successfully constructed against various antigens, for example, antibodies against IL-2 receptor as described in Queen et al., 1989, Proc. Natl. Acad. Sci. USA 86:10029; antibodies against the cell surface receptor CAMPATH as described in Riechmann et al., 1988, Nature 332:323; antibodies against hepatitis B in Co et al., 1991, Proc. Natl. Acad. Sci. USA 88:2869; as well as against viral antigens of the respiratory syncytial virus in Tempest et al., 1991, Bio-Technology 9:267. Humanized antibodies are most preferred for therapeutic use in humans.

Alternatively, techniques described for the production of single chain antibodies (U.S. Pat. No. 4,946,778; Bird, 1988, Science 242: 423-426; Huston et al., 1988, Proc. Natl. Acad. Sci. USA 85: 5879-5883; and Ward et al., 1989, Nature 334: 544-546) can be adapted to produce single chain antibodies against CD36 ligands, or portions thereof. Single chain antibodies are formed by linking the heavy and light chain fragments of the Fv region via an amino acid bridge, resulting in a single chain polypeptide.

Antibody fragments that recognize specific epitopes may be generated by known techniques. For example, such fragments include but are not limited to: the F(ab′)₂ fragments, which can be produced by pepsin digestion of the antibody molecule and the Fab fragments, which can be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity.

### 4.7 Assays for the Identification of Non-HSP-CD36 Ligands

The present invention uses methods for identifying compounds or molecules, other than those already known, that interact with the CD36 receptor. The purpose of identifying such compounds is to allow for the identification of such non-HSP-CD36 ligands so that extracorporeal methods may be used to remove such ligands from the blood. For example, antibodies may be produced against such compounds as described in Section 4.5 and attached to a chromatography column for use in extracorporeal procedures. The attached antibodies can be used to deplete non-HSP-CD36 ligands from the blood. Thus in vitro and in vivo assay systems described in the subsections below are used to identify compounds or compositions that interact with CD36.

Screening methodologies are used to identify small molecules, proteins and other compounds which interact with CD36. Such compounds may bind CD36 with differing affinities and may be useful in extracorporeal therapeutic applications in modulating the immune response.

Methods to screen potential agents for their ability to interact with CD36 ligand can be designed based on the receptor and its role in binding heat shock proteins such as gp96. CD36 protein, nucleic acids, and derivatives can be used in screening assays to detect molecules that specifically bind to CD36 ligands and derivatives. For example, recombinant cells expressing CD36 nucleic acids can be used to recombinantly produce CD36 in these assays, to screen for molecules that interfere with the binding of HSPs to CD36. Similar methods can be used to screen for molecules that bind to CD36 derivatives. Methods that can be used to carry out the foregoing are commonly known in the art.

### 4.7.1 CD36-Ligand Binding Assays

The screening assays, described herein, can be used to identify compounds and compositions, including peptides and organic, non-protein molecules that interact with CD36.

Thus, in a preferred embodiment, both naturally occurring and/or synthetic compounds (e.g., libraries of small molecules or peptides), may be screened for interacting with CD36. In another series of embodiments, cell lysates or tissue homogenates may be screened for proteins or other compounds which bind to one of the normal or mutant CD36 genes and CD36 polypeptides.

The screening assays described herein may be used to identify small molecules, peptides or proteins, or derivatives, analogs and fragments thereof, that interact with CD36. Such compounds will be removed from withdrawn blood by extracorporeal procedures.

The screening assays described herein are designed to detect compounds that modulate, i.e. interfere with ligand-receptor interactions. As described in detail below, such assays are finctional assays, such as binding assays, that can be adapted to a high-throughput screening methodologies.

Binding assays can be used to identify compounds that modulate the interaction between ligands and CD36. In one aspect of the invention the screens may be designed to identify compounds that disrupt the interaction between CD36 and a ligand.

Binding assays may be performed either as direct binding assays or as competition binding assays. In a direct binding assay, a test compound is tested for binding to CD36. Then, in a second step, the test compound is tested for its ability to modulate the ligand-CD36 interaction. Competition binding assays, on the other hand, assess the ability of a test compound to compete with a known ligand, i.e. an HSP, for binding to CD36.

In a direct binding assay, either the ligand and/or CD36 is contacted with a test compound under conditions that allow binding of the test compound to the ligand or the receptor. The binding may take place in solution or on a solid surface. Preferably, the test compound is previously labeled for detection. Any detectable compound may be used for labeling, such as but not limited to, a luminescent, fluorescent, or radioactive isotope or group containing same, or a nonisotopic label, such as an enzyme or dye. After a period of incubation sufficient for binding to take place, the reaction is exposed to conditions and manipulations that remove excess or non-specifically bound test compound. Typically, it involves washing with an appropriate buffer. Finally, the presence of a ligand-test compound or a CD36-test compound complex is detected.

In a competition binding assay, test compounds are assayed for their ability to disrupt or enhance the binding of the known ligand (e.g., HSP) to CD36. Labeled ligand (e.g., HSP) may be mixed with CD36 or fragment or derivative thereof, and placed under conditions in which the interaction between them would normally occur, with and without the addition of the test compound. The amount of labeled ligand (e.g., HSP) that binds CD36 may be compared to the amount bound in the presence or absence of test compound.

In a preferred embodiment, to facilitate complex formation and detection, the binding assay is carried out with one or more components immoblilized on a solid surface. In various embodiments, the solid support could be, but is not restricted to, polycarbonate, polystyrene, polypropylene, polyethlene, glass, nitrocellulose, dextran, nylon, polyacrylamide and agarose. The support configuration can include beads, membranes, microparticles, the interior surface of a reaction vessel such as a microtiter plate, test tube or other reaction vessel. The immobilization of CD36, or other component, can be achieved through covalent or non-covalent attachments. In one embodiment, the attachment may be indirect, i.e. through an attached antibody. In another embodiment, CD36 and negative controls are tagged with an epitope, such as glutathione S-transferase (GST) so that the attachment to the solid surface can be mediated by a commercially available antibody such as anti-GST (Santa Cruz Biotechnology).

For example, such an affinity binding assay may be performed using CD36 which is immobilized to a solid support. Typically, the non-mobilized component of the binding reaction, in this case either ligand (e.g., HSP) or the test compound, is labeled to enable detection. A variety of labeling methods are available and may be used, such as luminescent, chromophore, fluorescent, or radioactive isotope or group containing same, and nonisotopic labels, such as enzymes or dyes. In a preferred embodiment, the test compound is labeled with a fluorophore such as fluorescein isothiocyanate (FITC, available from Sigma Chemicals, St. Louis).

The labeled test compounds, or ligand (e.g., HSP) plus test compounds, are then allowed to contact with the solid support, under conditions that allow specific binding to occur. After the binding reaction has taken place, unbound and non-specifically bound test compounds are separated by means of washing the surface. Attachment of the binding partner to the solid phase can be accomplished in various ways known to those skilled in the art, including but not limited to chemical cross-linking, non-specific adhesion to a plastic surface, interaction with an antibody attached to the solid phase, interaction between a ligand attached to the binding partner (such as biotin) and a ligand-binding protein (such as avidin or streptavidin) attached to the solid phase, and so on.

Finally, the label remaining on the solid surface may be detected by any detection method known in the art. For example, if the test compound is labeled with a fluorophore, a fluorimeter may be used to detect complexes.

Preferably, CD36 is added to binding assays in the form of intact cells that express CD36, or isolated membranes containing CD36. Thus, direct binding to CD36 or the ability of a test compound to modulate a ligand-CD36 complex (e.g., HSP-CD36 complex) may be assayed in intact cells in culture or in animal models in the presence and absence of the test compound. A labeled ligand (e.g., HSP) may be mixed with cells that express CD36, or to crude extracts obtained from such cells, and the test compound may be added. Isolated membranes may be used to identify compounds that interact with CD36. For example, in a typical experiment using isolated membranes, cells may be genetically engineered to express CD36. Membranes can be harvested by standard techniques and used in an in vitro binding assay. Labeled ligand (e.g., ¹²⁵I-labeled HSP) is bound to the membranes and assayed for specific activity; specific binding is determined by comparison with binding assays performed in the presence of excess unlabeled (cold) ligand. Alternatively, soluble CD36 may be recombinantly expressed and utilized in non-cell based assays to identify compounds that bind to CD36. The recombinantly expressed CD36 polypeptides or fusion proteins containing the extracellular domain (ECD) of CD36, or one or more subdomains thereof, can be used in the non-cell based screening assays. Alternatively, peptides corresponding to one or more of the CDs of CD36, or fusion proteins containing one or more of the CDs of CD36 can be used in non-cell based assay systems to identify compounds that bind to the cytoplasmic portion of CD36; such compounds may be useful to modulate the signal transduction pathway of CD36. In non-cell based assays the recombinantly expressed CD36 is attached to a solid substrate such as a test tube, microtiter well or a column, by means well known to those in the art (see Ausubel et al., supra). The test compounds are then assayed for their ability to bind to CD36.

Alternatively, the binding reaction may be carried out in solution. In this assay, the labeled component is allowed to interact with its binding partner(s) in solution. If the size differences between the labeled component and its binding partner(s) permit such a separation, the separation can be achieved by passing the products of the binding reaction through an ultrafilter whose pores allow passage of unbound labeled component but not of its binding partner(s) or of labeled component bound to its partner(s). Separation can also be achieved using any reagent capable of capturing a binding partner of the labeled component from solution, such as an antibody against the binding partner, a ligand-binding protein which can interact with a ligand previously attached to the binding partner, and so on.

In one embodiment, for example, a phage library can be screened by passing phage from a continuous phage display library through a column containing purified CD36, or derivative, analog, fragment, or domain, thereof, linked to a solid phase, such as plastic beads. By altering the stringency of the washing buffer, it is possible to enrich for phage that express peptides with high affinity for CD36. Phage isolated from the column can be cloned and the affinities of the short peptides can be measured directly. Sequences for more than one oligonucleotide can be combined to test for even higher affinity binding to CD36. Knowing which amino acid sequences confer the strongest binding to CD36, computer models can be used to identify the molecular contacts between CD36 and the test compound. This will allow the design of non-protein compounds which mimic those contacts. Such a compound may have the same activity of the peptide and can be used therapeutically, having the advantage of being efficient and less costly to produce.

In another specific embodiment of this aspect of the invention, the solid support is membranes containing CD36 attached to a microtiter dish. Test compounds, for example, cells that express library members are cultivated under conditions that allow expression of the library members in the microtiter dish. Library members that bind to the protein (or nucleic acid or derivative) are harvested. Such methods, are described by way of example in Panmley and Smith, 1988, Gene 73:305-318; Fowlkes et al, 1992, BioTechniques 13:422427; PCT Publication No. WO 94/18318; and in references cited hereinabove.

In another embodiment of the present invention, interactions between CD36 or ligand (e.g., HSP) and a test compound may be assayed in vitro. Known or unknown molecules are assayed for specific binding to CD36 nucleic acids, proteins, or derivatives under conditions conducive to binding, and then molecules that specifically bind to CD36 are identified. The two components can be measured in a variety of ways. One approach is to label one of the components with an easily detectable label, place it together with a test component(s) under conditions that allow binding to occur, perform a separation step which separates bound labeled component from unbound labeled component, and then measure the amount of bound component. In one embodiment, CD36 can be labeled and added to a test agent, using conditions that allow binding to occur. Binding of the test agent can be determined using polyacrylamide gel analysis to compare complexes formed in the presence and absence of the test agent.

In yet another embodiment, binding of ligand (e.g., HSP) to CD36 may be assayed in intact cells in animal models. A labeled ligand (e.g., HSP) may be administered directly to an animal, with and without a test compound. Signal transduction stimulation of the ligand (e.g., HSP) may be measured in the presence and the absence of test compound. For these assays, host cells to which the test compound is added may be genetically engineered to express CD36 and/or ligand (e.g., HSP), which may be transient, induced or constitutive, or stable. For the purposes of the screening methods of the present invention, a wide variety of host cells may be used including, but not limited to, tissue culture cells, mammalian cells, yeast cells, and bacteria. Mammalian cells such as macrophages or other cells that express CD36, i.e., cells of the monocytic lineage, liver parenchymal cells, fibroblasts, keratinocytes, neuronal cells, and placental syncytiotrophoblasts, may be a preferred cell type in which to carry out the assays of the present invention. Bacteria and yeast are relatively easy to cultivate but process proteins differently than mammalian cells.

### 4.7.2 Compounds That can be Screened for Use in Extracorporeal Immunotherapy

The screening assays described herein may be used to identify small molecules, peptides or proteins, or derivatives, analogs and fragments thereof, that interact with non-HSP-CD36 ligands for use as non-HSP-CD36 ligand binding molecules in extracorporeal immunotherapy. These molecules can be attached to a chromatography column for use in extracorporeal procedures. The compounds that may be screened in accordance with the invention include, but are not limited to small molecules, peptides, antibodies and fragments thereof, and other organic compounds (e.g., peptidomimetics) as well as small molecules, peptides, antibodies or fragments thereof, and other organic compounds. Such molecules may be used in the present invention by attaching them to an affinity column and allowing extracted blood to pass through the column. The molecules will bind to the non-HSP-CD36 ligand and deplete the blood of the non-HSP-CD36 ligand.

Compounds that may be used for screening include, but are not limited to, peptides such as, for example, soluble peptides, including but not limited to members of random peptide libraries; (see, e.g., Lam et al., 1991, Nature 354:82-84; Houghten et al., 1991, Nature 354:84-86), and combinatorial chemistry-derived molecular library made of D- and/or L-configuration amino acids, phosphopeptides (including, but not limited to, members of random or partially degenerate, directed phosphopeptide libraries; see, e.g., Songyang et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), and small organic or inorganic molecules.

In one embodiment of the present invention, peptide libraries may be used as a source of test compounds that can be used in the present invention to deplete withdrawn blood of a non-HSP-CD36 ligand. Diversity libraries, such as random or combinatorial peptide or nonpeptide libraries can be screened for molecules that specifically bind to CD36 ligands (other than HSPs). Many libraries are known in the art that can be used, e.g., chemically synthesized libraries, recombinant (e.g., phage display libraries), and in vitro translationbased libraries.

Examples of chemically synthesized libraries are described in Fodor et al., 1991, Science 251:767-773; Houghten et al., 1991, Nature 354:84-86; Lam et al., 1991, Nature 354:82-84; Medynski, 1994, Bio/Technology 12:709-710; Gallop et al., 1994, J. Medicinal Chemistry 37(9):1233-1251; Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al., 1993, Proc. Natl. Acad. Sci. USA 90:11708-11712; PCT Publication No. WO 93/20242; and Brenner and Lerner, 1992, Proc. Natl. Acad. Sci. USA 89:5381-5383.

Examples of phage display libraries are described in Scott & Smith, 1990, Science 249:386-390; Devlin et al., 1990, Science, 249:404-406; Christian et al., 1992, J. Mol. Biol. 227:711-718; Lenstra, 1992, J. Immunol. Meth. 152:149-157; Kay et al., 1993, Gene 128:59-65; and PCT Publication No. WO 94/18318 dated Aug. 18, 1994.

By way of examples of nonpeptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) can also be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142).

Screening the libraries can be accomplished by any of a variety of commonly known methods. See, e.g., the following references, which disclose screening of peptide libraries: Parmley & Smith, 1989, Adv. Exp. Med. Biol. 251:215-218; Scott & Smith, 1990, Science 249:386-390; Fowlkes et al., 1992; BioTechniques 13:422-427; ; Oldenburg et al, 1992, Proc. Natl. Acad. Sci. USA 89:5393-5397; Yu et al., 1994, Cell 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al.; Rebar & Pabo, 1993, Science 263:671-673; and PCT Publication No. WO 94/18318.

In another embodiment of the present invention, the screening may be performed by adding the labeled CD36 ligand to in vitro translation systems such as a rabbit reticulocyte lysate (RRL) system and then proceeding with in vitro priming reaction. In vitro translationbased libraries include but are not limited to those described in PCT Publication No. WO 91/05058 dated Apr. 18, 1991; and Mattheakis et al., 1994, Proc. Natl. Acad. Sci. USA 91:9022-9026.

Compounds that can be tested and identified methods described herein can include, but are not limited to, compounds obtained from any commercial source, including Aldrich (Milwaukee, Wis. 53233), Sigma Chemical (St. Louis, Miss.), Fluka Chemie AG (Buchs, Switzerland) Fluka Chemical Corp. (Ronkonkoma, N.Y.;), Eastman Chemical Company, Fine Chemicals (Kingsport, Tenn.), Boehringer Mannheim GmbH (Mannheim, Germany), Takasago (Rockleigh, N.J.), SST Corporation (Clifton, N.J.), Ferro (Zachary, La. 70791), Riedel-deHaen Aktiengesellschaft (Seelze, Germany), PPG Industries Inc., Fine Chemicals (Pittsburgh, Pa. 15272). Further any kind of natural products may be screened using the methods of the invention, including microbial, fungal, plant or animal extracts.

Furthermore, diversity libraries of test compounds, including small molecule test compounds, may be utilized. For example, libraries may be commercially obtained from Specs and BioSpecs B.V. (Rijswijk, The Netherlands), Chembridge Corporation (San Diego, Calif.), Contract Service Company (Dolgoprudny, Moscow Region, Russia), Comgenex USA Inc. (Princeton, N.J.), Maybridge Chemicals Ltd. (Cornwall PL34 OHW, United Kingdom), and Asinex (Moscow, Russia).

Still further, combinatorial library methods known in the art, can be utilized, including, but not limited to: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam,1997, Anticancer Drug Des.12:145). Combinatorial libraries of test compounds, including small molecule test compounds, can be utilized, and may, for example, be generated as disclosed in Eichler & Houghten, 1995, Mol. Med. Today 1:174-180; Dolle, 1997, Mol. Divers. 2:223-236; and Lam, 1997, Anticancer Drug Des. 12:145-167.

Examples of methods for the synthesis of molecular libraries can be found in the art, for example in: DeWitt et al, 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented in solution (e.g., Houghten, 1992, BioTechniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) orphage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310).

Screening the libraries can be accomplished by any of a variety of commonly known methods. See, e.g., the following references, which disclose screening of peptide libraries: Parmley & Smith, 1989, Adv. Exp. Med. Biol. 251:215-218; Scott & Smith, 1990, Science 249:386-390; Fowlkes et al, 1992; BioTechniques 13:422-427; Oldenburg et al., 1992, Proc. Natl. Acad. Sci. USA 89:5393-5397; Yu et al, 1994, Cell 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al.; Rebar & Pabo, 1993, Science 263:671-673; and PCT Publication No. WO 94/18318.

### 4.8 Identification of Fragments of CD36 and/or CD36 Ligands Useful for Immunotherapy

The invention also encompasses methods for identifying CD36 ligand-binding fragments, and analogs, muteins, or derivatives thereof, which are capable of binding to CD36 ligands for use in extracorporeal immunotherapy. Such ligand-binding CD36 fragments can be used in extracorporeal therapy by attaching such fragments to an affinity column and allowing withdrawn blood to pass through the column. The CD36 ligand-binding fragments will deplete the blood of the non-HSP-CD36 ligand. In one embodiment, such a method for identifying a CD36 fragment capable of binding a CD36 ligand other than an HSP comprises the steps of: (a) contacting a CD36 ligand other than an HSP with one or more CD36 fragments; and (b) identifying a CD36 polypeptide fragment which specifically binds to the CD36 ligand.

CD36 fragments, analogs, muteins, and derivatives and/or ligand fragments, analogs, muteins, and derivatives of the invention may be produced by recombinant DNA techniques, synthetic methods, or by enzymatic or chemical cleavage of native CD36 and/or ligands.

Any eukaryotic cell may serve as the nucleic acid source for obtaining the coding region of a non-HSP-CD36 ligand gene. Nucleic acid sequences encoding ligand and or CD36 can be isolated from vertebrate, mammalian, as well as primate sources, including humans. Amino acid sequences and nucleotide sequences of naturally occurring ligands and CD36 are generally available in sequence databases, such as Genbank.

The DNA may be obtained by standard procedures known in the art by DNA amplification or molecular cloning directly from a tissue, cell culture, or cloned DNA (e.g., a DNA “library”). Clones derived from genomic DNA may contain regulatory and intron DNA regions in addition to coding regions; clones derived from cDNA will contain only exon sequences. In a preferred embodiment, DNA can be amplified from genomic or cDNA by polymerase chain reaction (PCR) amplification using primers designed from the known sequence of an ligand. The polymerase chain reaction (PCR) is commonly used for obtaining genes or gene fragments of interest. For example, a nucleotide sequence encoding a fragment of any desired length can be generated using PCR primers that flank the nucleotide sequence encoding the peptide-binding domain. Alternatively, a CD36 ligand or other CD36 ligand receptor gene sequence can be cleaved at appropriate sites with restriction endonuclease(s) if such sites are available, releasing a fragment of DNA encoding the peptide-binding domain. If convenient restriction sites are not available, they may be created in the appropriate positions by site-directed mutagenesis and/or DNA amplification methods known in the art (see, for example, Shankarappa et al., 1992, PCR Method Appl. 1:277-278). The DNA fragment that encodes a fragment of the ligand or CD36 gene is then isolated, and ligated into an appropriate expression vector, care being taken to ensure that the proper translation reading frame is maintained. Alternatives to isolating the genomic DNA include, but are not limited to, chemically synthesizing the gene sequence itself from a known sequence or making cDNA to the mRNA which encodes the ligand and/or CD36.

Any technique for mutagenesis known in the art can be used to modify individual nucleotides in a DNA sequence, for purpose of making amino acid substitution(s) in the expressed peptide sequence, or for creating/deleting restriction sites to facilitate further manipulations. Such techniques include but are not limited to, chemical mutagenesis, in vitro site-directed mutagenesis (Hutchinson, C., et al., 1978, J. Biol. Chem 253:6551), oligonucleotide-directed mutagenesis (Smith, 1985, Ann. Rev. Genet. 19:423-463; Hill et al., 1987, Methods Enzymol. 155:558-568), PCR-based overlap extension (Ho et aL., 1989, Gene 77:51-59), PCR-based megaprimer mutagenesis (Sarkar et al., 1990, Biotechniques, 8:404-407), etc. Modifications can be confirmed by double stranded dideoxy DNA sequencing.

An alternative to producing CD36 and/or CD36 ligand binding molecule fragments by recombinant techniques is peptide synthesis. For example, a peptide corresponding to a portion of CD36 and/or CD36 ligand binding molecule fragments comprising the substratebinding domain, or which binds peptides in vitro, can be synthesized by use of a peptide synthesizer. Conventional peptide synthesis may be used or other synthetic protocols well known in the art.

In addition, analogs and derivatives of CD36 and/or CD36 ligand binding molecule fragments can be chemically synthesized. Furthermore, if desired, nonclassical amino acids or chemical amino acid analogs can be introduced as a substitution or addition into CD36 and/or ligand sequence. Non-classical amino acids include but are not limited to the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general.

CD36 and/or CD36 ligand binding molecule fragments, or a mutant or derivative thereof, may be synthesized by solid-phase peptide synthesis using procedures similar to those described by Merrifield, 1963, J. Am. Chem. Soc., 85:2149. During synthesis, N-α-protected amino acids having protected side chains are added stepwise to a growing polypeptide chain linked by its C-terminal and to an insoluble polymeric support i.e., polystyrene beads. The peptides are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc which is acid labile and Fmoc which is base labile. Details of appropriate chemistries, resins, protecting groups, protected amino acids and reagents are well known in the art and so are not discussed in detail herein (See, Atherton, et al, 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting fragment is accomplished using conventional procedures, such as preparative HPLC using gel permeation, partition and/or ion exchange chromatography. The choice of appropriate matrices and buffers are well known in the art and so are not described in detail herein.

In an alternative embodiment, fragments of CD36 and/or CD36 ligand binding molecule fragments may be obtained by chemical or enzymatic cleavage of native or recombinant CD36 and/or CD36 ligand binding molecule fragments. Specific chemical cleavage can be performed by cyanogen bromide, NaBH₄, acetylation, formylation, oxidation, reduction, metabolic synthesis in the presence of tunicamycin, etc.. Endoproteases that cleave at specific sites can also be used. Such proteases are known in the art, including, but not limited to, trypsin, α-chymotrypsin, V8 protease, papain, and proteinase K (see Ausubel et al., (eds.), in “Current Protocols in Molecular Biology”, Greene Publishing Associates and Wiley Interscience, New York, 17.4.6-17.4.8). CD36 and/or CD36 ligand binding molecule fragments of interest can be examined for the recognition sites of these proteases. An enzyme is chosen which can release a peptide-binding domain or peptidebinding fragment. CD36 and/or CD36 ligand binding molecule is then incubated with the protease, under conditions that allow digestion by the protease and release of the specifically designated peptide-binding fragments. Alternatively, such protease digestions can be carried out blindly, i.e., not knowing which digestion product will contain the peptide-binding domain, using specific or general specificity proteases, such as proteinase K or pronase.

Once a fragment is prepared, the digestion products may be purified as described above, and subsequently tested for the ability to bind peptide or for immunogenicity.

### 4.9 Target Infectious Disease

The infectious diseases that can be treated or prevented using the methods of the present invention include those caused by intracellular pathogens such as viruses, bacteria, protozoans, and intracellular parasites. Viruses include, but are not limited to viral diseases such as those caused by hepatitis type B virus, parvoviruses, such as adeno-associated virus and cytomegalovirus, papovaviruses such as papilloma virus, polyoma viruses, and SV40, adenoviruses, herpes viruses such as herpes simplex type I (HSV-I), herpes simplex type II (HSV-II), and Epstein-Barr virus, poxviruses, such as variola (smallpox) and vaccinia virus, RNA viruses, including but not limited to human immunodeficiency virus type I (HIV-I), human immunodeficiency virus type II (HIV-II), human T-cell lymphotropic virus type I (HTLV-I), and human T-cell lymphotropic virus type II (HTLV-II); influenza virus, measles virus, rabies virus, Sendai virus, picomaviruses such as poliomyelitis virus, coxsackieviruses, rhinoviruses, reoviruses, togaviruses such as rubella virus (German measles) and Semliki forest virus, arboviruses, and hepatitis type A virus.

In another embodiment, bacterial infections can be treated or prevented such as, but not limited to disorders caused by pathogenic bacteria including, but not limited to, Streptococcus pyogenes, Streptococcus pneumoniae, Neisseria gonorrhoea, Neisseria meningitidis, Corynebacterium diphtheriae, Clostridium botulinum, Clostridium perfringens, Clostridium tetani, Haemophilus influenzae, Klebsiella pneumoniae, Klebsiella ozaenae, Klebsiella rhinoscleromotis, Staphylococcus aureus, Vibrio cholerae, Escherichia coli, Pseudomonas aeruginosa, Campylobacter (Vibrio) fetus, Campylobacterjejuni, Aeromonas hydrophila, Bacillus cereus, Edwardsiella tarda, Yersinia enterocolitica, Yersinia pestis, Yersinia pseudotuberculosis, Shigella dysenteriae, Shigellaflexneri, Shigella sonnei, Salmonella typhuimurium, Salmonella typhii, Treponema pallidum, Treponema pertenue, Treponema carateneum, Borrelia vincentii, Borrelia burgdorferi, Leptospira icterohemorrhagiae, Mycobacterium tuberculosis, Toxoplasma gondii, Pneumocystis carinii, Francisella tularensis, Brucella abortus, Brucella suis, Brucella melitensis, Mycoplasma spp., Rickettsia prowazeki, Rickettsia tsutsugumushi, Chlamydia spp., and Helicobacter pylori.

In another preferred embodiment, the methods can be used to treat or prevent infections caused by pathogenic protozoans such as, but not limited to, Entomoeba histolytica, Trichomonas tenas, Trichomonas hominis, Trichomonas vaginalis, Trypanosoma gambiense, Trypanosoma rhodesiense, Trypanosoma cruzi, Leishmania donovani, Leishmania tropica, Leishmania braziliensis, Pnieumocystis pneumonia, Plasmodium vivax, Plasmodium falciparum, and Plasmodium malaria.

### 4.10 Target Proliferative Cell Disorders

With respect to specific proliferative and oncogenic disease associated with HSPCD36 activity, the diseases that can be treated or prevented by the methods of the present invention include, but are not limited to: human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

Diseases and disorders involving a deficiency in cell proliferation or in which cell proliferation is desired for treatment or prevention, and that can be treated or prevented by inhibiting CD36 function, include but are not limited to degenerative disorders, growth deficiencies, hypoproliferative disorders, physical trauma, lesions, and wounds; for example, to promote wound healing, or to promote regeneration in degenerated, lesioned or injured tissues, etc.

The invention is not to be limited in scope by the specific embodiments described which are intended as single illustrations of individual aspects of the invention, and functionally equivalent methods and components are within the scope of the invention. Indeed various modifications of the invention, in addition to those shown and described herein will become apparent to those skilled in the art from the foregoing description and accompanying drawings. Such modifications are intended to fall within the scope of the appended claims.

All references cited herein, including patent applications, patents, and other publications, are incorporated by reference herein in their entireties for all purposes. The invention is not to be limited in scope by the specific embodiments described which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

All references cited herein, including patent applications, patents, and other publications, are incorporated by reference herein in their entireties for all purposes.

What is claimed is:

1. A method for stimulating an immune response in a patient comprising administering to said patient blood which has been withdrawn from said patient and treated to remove a CD36 ligand.

2. The method of claim 1 further comprising administering to said patient a heat shock protein or a heat shock protein-antigenic peptide complex.

3. A method for stimulating an immune response in a patient comprising:
(a) removing a CD36 ligand from blood withdrawn from said patient; and
(b) returning at least a portion of the CD36 ligand-depleted blood to said patient.

4. A method for stimulating an immune response in a patient comprising:
(a) withdrawing blood from said patient;
(b) removing a CD36 ligand from said blood; and
(c) returning at least a portion of the CD36 ligand-depleted blood to said patient.

5. The method of claim 4 further comprising after step (a) and before step (c) the step of adding a heat shock protein or a heat shock protein-antigenic peptide complex to said blood.

6. The method of claims 3 or 4 wherein removing a CD36 ligand from the blood comprises the step of contacting the blood with a solid phase attached to a CD36 ligand-binding molecule for a time period and under conditions sufficient to allow binding of CD36 ligand to the CD36 ligand-binding molecule solid phase.

7. The method of claim 6 wherein the CD36 ligand-binding molecule is CD36, or a fragment thereof.

8. The method of claim 6 wherein said CD36 ligand-binding molecule does not bind a heat shock protein.

9. The method of claim 8 wherein the CD36 ligand-binding molecule is an CD36 ligand-specific antibody, or a fragment thereof.

10. The method of claims 3 or 4 wherein an apheresis system is used in said removing step.

11. The method of claim 4 wherein blood is withdrawn manually in said withdrawing step.

12. The method of claim 3 or 4 wherein said removing step comprises separating the plasma from said blood and treating said plasma to remove said CD36 ligand.

13. The method of claim 1 wherein said blood is administered to said patient by syringe.

14. The method of claim 1 wherein said blood is administered to said patient by an intravenous drip.

15. The method of claim 3 or 4 wherein said blood is returned to said patient by syringe.

16. The method of claim 3 or 4 wherein said blood is returned to said patient by an intravenous drip.

17. A kit comprising in one or more containers a solid phase chromatography column with a purified CD36 ligand binding molecule attached thereto, such that withdrawn blood can be run over the column to deplete the blood of a CD36 ligand.

18. The kit of claim 17 wherein the CD36 ligand binding molecule does not bind heat shock proteins.

19. The method of claim 1, 3, or 4 wherein the CD36 ligand is a lipoprotein complex, thrombospondin 1, P. falciparum erythrocyte membrane protein 1, or a phospholipid.
